(19) United States
(12) Patent Application Publication
(10) Pub. No.: US 2007/0287690 A1
(43) Pub. Date: Dec. 13, 2007

(54) COMPOSITIONS AND METHODS COMPRISING A THIAZIDE DIURETIC AND A MINERALOCORTICOID INHIBITOR

(76) Inventors: Lawrence Solomon, Boca Raton, FL (US); Michael Weber, New York, NY (US)

Correspondence Address: ACCU-BREAK TECHNOLOGIES, INC., 1000 SOUTH PINE ISLAND ROAD, SUITE 230, PLANTATION, FL, 33324, US

(21) Appl. No.: 11/760,198

(22) Filed: Jun. 8, 2007

Related U.S. Application Data
(60) Provisional application No. 60/811,926, filed on Jun. 8, 2006.

(51) Int. Cl. A61K 31/57; A61K 31/549

(52) U.S. Cl. 514/171; 514/223.5

(57) ABSTRACT

The invention relates to low dose combinations of a thiazide diuretic and a mineralocorticoid inhibitor and methods for the treatment of hypertension and related disorders. Methods and compositions for administering a thiazide diuretic, such as chlorthalidone, and an mineralocorticoid inhibitor such as spironolactone or eplerenone in a dose range of between 3 mg and 12 mg or eplerenone in a dose range of between 3 mg and 25 mg each,

### CROSS-REFERENCE TO RELATED APPLICATION

This is a continuation of U.S. Provisional Application Ser. No. 60/811,926 filed Jun. 8, 2006.

### FIELD OF THE INVENTION

The invention relates to a low dose combination of a thiazide diuretic and a mineralocorticoid inhibitor for the treatment or prevention of cardiovascular diseases, such as hypertension, heart failure, and related disorders. The low dose combination is effective and has reduced side-effects compared to conventional dosages of its component drugs.

### BACKGROUND OF THE INVENTION

Hypertension and heart failure are characterized by increased vascular volume, inadequate sodium (Na⁺) excretion, and increased circulating aldosterone levels. Aldosterone is a mineralocorticoid that promotes Na⁺ and water resorption at the expense of potassium (K⁺) and magnesium (Mg⁺) excretion. Chronic elevation of the plasma aldosterone level that is inappropriate relative to dietary Na⁻ intake can have adverse consequences on tie structure and function of the cardiovascular system and can contribute to the clinical consequences of hypertension and heart failure.

Diuretic treatment of hypertension and heart failure is effective across a wide range of patients and also protects hypertensive patients from strokes and heart failure (Antihypertensive and Lipid-Lowering to Prevent Elheart Attack Trial (ALLHAT)). For that reason, the Joint National 25 Committee on Prevention, Detection, Evaluation, and Treatment of High Blood Pressure guidelines recommends the use of diuretics as a suitable therapy for a wide range of patients.

However, despite these data and recommendations, diuretics—particularly as single agent therapy—are not widely used because of their adverse side effects. One side effect of thiazide diuretics (for example, chlorthalidone) is the loss of potassium resulting in low plasma potassium concentrations, a condition known as hypokalemia. In the past, attempts have been made to compensate for this problem by simultaneously prescribing other types of diuretics, such as spironolactone. Spironolactone is a mineralocorticoid receptor antagonist that blocks mineralocorticoid action by competitively binding to the aldosterone receptor, thereby helping to eliminate sodium while conserving potassium levels. However, spironiolactone at the available doses (25 mg, 50 mg and 100 mg) is not completely selective in its aldosterone blocking effects, but blocks other steroid receptors as well. This has the effect of causing reduced sexual function and gynecomastia (breast swelling), which is upsetting to male patients and a cause of significant discomfort in female patients.

A limited number of combination drug products, using at least one diuretic have been marketed. These are believed to be limited to potassium-sparing diuretics and potassium-wasting diuretics. These combinations include:
1) spironolactone/hydroclhlorothiazide (“H;CTZ”) (Aldactazide);
2) spironolactone/furosemide (Lasilactone®);
3) triamterene/HCTZ (Dyazide® and Maxzide®); and
4) amiloride/HCTZ (Moduretic®)

Other examples of high-dose combination products are also available. It is believed that in the triamterene and amiloride combinations with HCTZ (#'s 3 and 4 above), the potassium-sparing diuretics do not contribute to the antihypertensive efficacy of the combination, but are present only to mitigate potassium loss often induced by HCTZ. For the spironolactone-HCTZ combination (#1 above), it is believed that the spironolactone is of modest antihypertensive efficacy in comparison to the HCTZ moiety. No data have been identified relating to the efficacy of the spironolactone-furosemide combination (#2 above).

WO 96(24358 discloses co-administering or co-formulating spironolactone and a thiazide-type diuretic throughout a wide variety of dosages. However, no antihypertensive combination of a thiazide diuretic, such as HCTZ or chlorthalidone has been marketed that can provide an unexpected advance in the art such as a fully additive or synergistic effect on blood pressure.

A need therefore exists for an efficacious diuretic-based treatment for hypertension and heart failure that minimizes or completely avoids the adverse effects frequently encountered with conventional diuretic therapies.

### SUMMARY OF THE INVENTION

The subject invention concerns a composition and method of use which comprises a low dose combination of a thiazide diuretic and a mineralocorticoid inhibitor for the treatment or prevention of cardiovascular diseases, such as hypertension, heart failures and related disorders. The low dose combination advantageously provides an effective treatment for such conditions and can provide reduced side-effects to a patient being administered the low dose combination, as compared to conventional dosages of its component drugs. The combination can comprise both drugs co-formulated in a single dosage form, or can comprise the two drugs co-packaged for co-administration. Co-administration can include administering the drugs together or separately, in any order, simultaneously or at different times within 24 hours of the other.

One object of the subject invention includes providing a composition and method which can result in a synergistic or fully additive effect by use of the two component drugs in combination. Preferred combinations include all thiazide-type diuretics, and more preferably, spironolactone, in combination with HCTZ. Chlorthalidone (also spelled chlortalidone) may also be combined with a mineralocorticoid inhibitor, e.g., spironolactone or eplerenone.

Another object of the subject invention is a composition and treatment method which comprises administering to a patient a combination drug product in accordance with the subject invention, which advantageously achieves potassium neutrality in the patient. Yet another object of the invention includes a composition and method wherein equal contribution to the hypotensive effect in a patient is achieved by the two components of the combination.

Another preferred object of the invention involves methods of treatment with a combination, or co-administration of, a thiazide-type diuretic and an aldosterone antagonist.

The invention also involves the novel discovery that a thiazide-type diuretic and an aldosterone antagonist combination product (or co-administration) has a unique set of doses, or ratio of doses, that achieve specific therapeutic benefits that cannot be predicted by the current art. These benefits include optimal potassium balance and equal contribution to the antihypertensive activity of the combination from each moiety.

Optimal potassium balance achieved by a combination product (or co-administration of the component drugs of the combination) can be shown administering the composition of the subject invention to different groups of patients. One group comprises patients not on another drug known to affect potassium blood levels, such as angiotensill converting enzyme (ACE) inhibitors, angiotensin receptor blockers (ARBs), or beta blockers (BBs). A different group can include diabetics, who are prone to relative hyperkalemia. A third group can include patients receiving serum/plasma potassium-altering drugs. Evidence from a specific drug combination of the subject invention of either potassium neutrality, or achieving potassium balance in an optimal manner in these defined populations is a novel and unexpected finding.

The invention provides low dose combinations and pharmaceutical compositions of a thiazide diuretic and a mineralocorticoid inhibitor for the treatment or prevention of cardiovascular diseases, such as hypertension, heart failure and related disorders. The low dosage does not disrupt a patient's normal electrolyte and water-retention balance, due to the compensating effects of the tow components, yet provides efficacy similar to that observed with either one given in full conventional doses.

In one aspect, the invention provides compositions comprising a dosage equivalent of about 3 mg to about 12 mg of a thiazide diuretic or the mineralocorticoid inhibitor, spironolactone. In another embodiment, the invention provides a composition comprising a dosage equivalent of about 3 mg to about 25 mg of eplerenone rather than spironolactone. Such low doses of individual diuretics may be used in various combinations in the practice of the methods of the invention. In an embodiment, the composition also comprises a pharmaceutically acceptable carrier.

In another embodiment, the invention provides compositions comprising a dosage equivalent of about 3 mg to about 12 mg of a thiazide diuretic and about 3 mg to about 12 mg of the mineralocorticoid inhibitor, spironolactone. In another embodiment, the invention provides compositions comprising a dosage equivalent of about 3 mg to about 12 mg of a thiazide diuretic and about 3 mg to about 25 mg of eplerenone. In an embodiment, the composition also comprises a pharmaceutically acceptable carrier.

In an embodiment, the thiazide diuretic is chlorthalidone, hydrochlorothiazide and/or chlorothiazide.

In an embodiment, the mineralocorticoid inhibitor is a mineralocorticoid receptor antagonist, such as, for example, spironolactone and eplerenone.

In another aspect, the invention provides methods for treating hypertension or heart failure by administering to a patient a dosage amount equivalent to about 3 mg to about 12 mg of a thiazide diuretic and about 3 mg to about 12 mg of the mineralocorticoid inhibitor, spironolactone. In another embodiment, the invention provides pharmaceutical compositions comprising a dosage equivalent of about 3 mg to about 12 mg of a thiazide diuretic and about 3 mg to about 25 mg of eplerenone. In an embodiment, the dosage also comprises a pharmaceutically acceptable carrier.

In an embodiment, the thiazide diuretic is administered simultaneously with the mineralocorticoid inhibitor. In another embodiment, the thiazide diuretic is administered separately from the mineralocorticoid inhibitor, for example, within about 48 hours of each other.

In an embodiment, the thiazide diuretic and the mineralocorticoid inhibitor are administered orally. However, any mode of administration is contemplated, such as, for example, intravenous, intramuscular, topical, inhalation, rectal, and/or ocular administration.

### BRIEF DESCRIPTION OF TI IE DRAWINGS

The foregoing and other objects, features and advantages of the present invention, as well as the invention itself, will be more fully understood from the following description of preferred embodiments when read together with the accompanying drawings in which:

FIG. 1 provides the outline of the study design for a clinical trial designed to test the efficacy and tolerability of low dose combinations of a thiazide-type diuretic and a mineralocorticoid receptor antagonist when compared with full conventional doses of the individual agents.

### DETAILED DESCRIPTION OF TIEFNVENTION

The invention relates to compositions and methods for the treatment or prevention of circulatory disorders, including cardiovascular disorders such as hypertension and congestive heart failure, by a therapy comprising a therapeutically-effective low dose (between about 3 mg to about 12 mg each) of a thiazide diuretic and a mineralocorticoid inhibitor, such an aldosterone inhibitor. In an embodiment, eplerenone is formulated and administered in a dosage of between about 3 mg and about 25 mg. These doses are substantially lower than those normally used in clinical situations and are intended to avoid the adverse side effects of traditional doses (25 mg to 100 mg).

In addition to thiazides, the diuretic used in combination with a mineralocorticoid inhibitor may be selected from several known classes: thiazide-type diuretics (related sulfonamides), potassium-sparing diuretics, loop diuretics and organic mercurial diuretics.

Examples of thiazides are bendroflumethiazide (e.g., Naturctin-5), benzthiazide, chlorothiazide, cyclothiazide (e.g., Anhydron, Fluidil), hydrochliorothiazide (e.g., Esidrix, Hydro-D, Microzide, Zide,), hydroflumethiazide (e.g., Ducardin, Saluron), methyclothiazide (e.g., Aquatensen, Enduron), polythiazide (e.g., Renese) and trichlormethiazide (e.g., Metahydrin, Naqua, Trichlorex, Trichlormas).

Examples of related sulfonamides (thiazide-type) are chlorthalidone (e.g., Hygroton, Thalitone), quinethazone (e.g., Hydromox) and metolazone (e.g., Diulo, Mykrox, Zaroxolyn). As used herein, the phrase “thiazide” diuretic includes “thiazide-type” or “thiazide-like” diuretics. Indapamide (e.g. Lozol) is a unique non-thiazide diuretic

Examples of potassium-sparing diuretics that arc also aldosterone receptor antagonists are spironolactone (e.g., Aldactone; Spironol) and eplerenone (e.g., Inspra).

Examples of loop diuretics, i.e., diuretics acting in the ascending limb of the loop of Henle of the kidney, are furosemide (e.g., Lasix), ethynacrylic acid and torsemide (e.g., Demadex).

Examples of organic mercurial diuretics are mercaptomerin sodium, merethoxylline procaine and mersalyl with theophylline.

The compositions and methods of the invention are also useful, for example, for preventing or retarding, in a hypertensive subject, the development of heart attacks, congestive heart failure and stroke, and for reducing mortality.

The compositions and methods of the invention can also be useful in the treatment of any of a number of hypertensive disorders, for example, congenital or acquired endocrine hypertensive disorders such as Apparent Mineralocorticoid Excess (AME), Congenital Adrenal Hyperplasia (CAH), Familial Glucocorticoid Resistance (FGR), Liddle Syndrome, Geller Syndrome, and Pseudohypoaldosteronism Type II, for example (Endocrine Hypertension; Apparent Mineralocorticoid Excess (AME) (HSD11B2) (February 2005) Reviews in Endocrinology; http://www.correlagen.com/diseasereviews/881.html).

The invention involves compositions that have been disclosed in a theoretical sense but that have had conflicting results in practice, as well as novel methods of administration.

Regarding synergy, such effect can be demonstrated for two anti-hypertensive agents according to the following:
1. Treat a patient with a dose X of drug A (but not with drug B), and adjust for placebo effect.
2. Treat with a dose Y of drug B (but not with drug A), and adjust for placebo effect.
3. Treat with a combination dose of or co-administer dose X of drug A and dose Y of drug B, and adjust for placebo effect.
4. If the placebo-adjusted BP effect of the combination (or co-administration) exceeds the sum of the placebo-adjusted effects of drug A alone plus drug B alone, the synergy has been demonstrated for that combination or co-administration.

Alternatively, synergy may be demonstrated by the following special case of the same dose of each of drug A and drug B being used together:
1. Treat a patient with a dose 2× of drug A (but not with drug B), adjust for placebo effect.
2. Treat with a dose 2× of drug B (same dose as that for drug A ), adjust for placebo effect.
3. Treat with a combination dose of or co-administer dose X of drug A and dose Y of drug B, adjust for placebo effect.
4. If the placebo-adjusted BP effect of the combination (or co-administration) exceeds either the placebo-adjusted effect of 2× of drug A alone, and the placebo-adjusted effect of 2× of drug B alone, then synergy has been demonstrated for that combination or co-administration.

Fully additive effect for the subject combination has also rarely if ever been shown for two anti-hypertensive agents given together, so that such a showing according to the claimed invention would be surprising as well as novel.

Other forms of synergy may exist, such as a synergistic effect relating to anti-fibrotic effect, edema reduction, regression of left ventricular hypertrophy, or extension of the duration of homodynamic effects.

In another embodiment of the invention, eplerenone plus HCTZ have been demonstrated to be non-additive when given in combination. The invention comprises a novel and surprising method of additive to treatment of hypertension utilizing those two drugs during co-administration.

Alternatively, or in addition to the synergistic or fully additive effect of the drug combinations discussed above, other desirable and unexpected effects can be achieved by the compositions or methods of the subject invention. For example, it is known that aldosterone antagonists can raise plasma and serum potassium levels, in contrast to thiazidetype diuretics, which can lower those parameters. It is not known, however, that there will be a definable combination of doses of particular agents that is superior to other combination of doses, especially in the lower dosing ranges where the metabolic effects of each type of agent are muted. In addition, the experience of Aldactazide and of the eplerenone plus HCTZ trial suggests that a thiazide-type diuretic co-administered with an aldosterone antagonist which demonstrates equal contribution of each agent to the blood pressure reduction (one of the desired goals of anti-hypertensive therapy according to the subject invention) is a surprising result.

Spironolactone's (“SP”) chemical name is 17-hydroxy-7a-mercapto-3-oxo-17α-pregn-4-ene-21-carboxylic acid γ-lactone acetate. This compound, its activities, modes of synthesis and purification are described in a number of U.S. patents including U.S. Pat. Nos. 3,013,012 and 4,529,811. Spironolactone is sold by GD Searle as “ALDACTONE”, in tablet dosage form at doses of 25 mg, 50 mg and 100 mg per tablet; and as a generic by Sandoz at 25 mg, by Purepac at 50 mg and 100 mg; and by Mylan and Mutual Pharm at 25 mg, 50 mg and 100 mg. Its empirical formula is C₂₄H₃₂O₄S and its structural formula is:

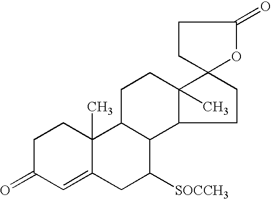

Eplerenone's chemical name is Pregn-4-ene-7,21-dicarboxylic acid, 9,11-epoxy-17-hydroxy-3-oxo-,γ-lactone, methyl ester, (7a,11a,17a)-. Eplerenone is a spironolactone-like aldosterone receptor antagonist that has a higher specificity for aldosterone receptors than spironolaetone. Eplerenone is sold as “INSPRA” at 25 mg and 50 mg. Its empirical formula is C₂₄H₃₀O₆ and its structural formula is:

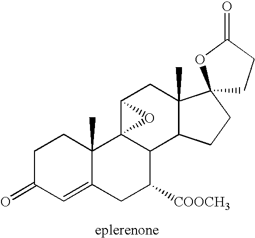

In addition, certain drugs have been identified that appear to block aldosterone synthesis and/or secretion by a more direct mechanism. These drugs include 15,16-methylene spirolactone compounds called Mespirenone (also called ZK 94679) and dethilolated Mespirenone (also called ZK 91587).

Hydrochlorothiazide's (HCTZ) chemical name is 6-chloro-3,4-dihydro-2H-1,2,4-benzothiadiazine-7-sulfonamide, 1,1-dioxide. HCTZ belongs to the benzothiadiazine class of thiazide diuretics. HCTZ is sold by Novartis as “ESIDRIX” at 25 mg and 50 mg, Abbott Labs as “ORETIC” at 50 mg, by Solvay as “ZIDE,” at 50 mg, and by Watson Labs as “MICROZIDE” at 12.5 mg and by Clonmell Healthcare, Purepac Pharma, Watson Labs, Sandoz, and EON at 25 mg and 50 mg; and Ivax, Mylan, and Vintage Pharms at 12.5 mg. Its empirical formula is C₇H₈ClN₃O₄S₂ and its structural formula is:

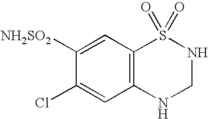

Chlorothiazide's chemical name is 6-chloro-2H,1,2,4-benzothia-diazine-7sulfamide 1, 1-dioxide. Chlorothiazide is sold by Merck as DIURIL as an injection: 500 mg base/vial suspension or oral: 250 mg/5 ml. Its empirical name is C₇H₆ClN₃O₄S₂ and its structural formula is:

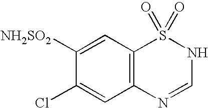

Chlorthalidone's (CLT) chemical name is 2-chloro-5-(1-hydroxy-3-oxo-1-isoindolinyl benzenesulfonamide. It's empirical formula is C₁₄H₁₁C₁N₂O₄S. Although commonly considered a thiazide, chlorthalidone is not a benzothiadiazine and is more appropriately called a thiazide-type diuretic. CTL is sold by Mylan and Pliva in tablet dosage form at doses of 25 mg and 50 mg and Monarch Pharmaceuticals at 15 mg.

It would be recognized by persons of ordinary skill in the art that the drugs named and referred to herein, also include their respective salts, isomers, polymorphs, derivatives and active metabolites or prodrugs.

Preparations for administration of the drugs may be suitably formulated to give controlled release of the active compound. For oral administration, the drugs may take the form of tablets or lozenges formulated in a conventional manner.

For administration by inhalation, the drugs for use according to the methods of the invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the drugs and a suitable powder base such as lactose or starch.

The drugs may be formulated for parenteral administration by injection, e.g., by bolus injection or continuous infusion. Formulations for injection may be presented in unit dosage form, e.g., in ampoules or in multi-dose containers, with an added preservative. The drugs may take such forms as suspensions, solutions or emulsions in oily or aqueous vehicles, and may contain formulation agents such as suspending, stabilizing and/or dispersing agents. Alternatively, the drugs may be in powder form for constitution with a suitable vehicle, e.g., sterile pyrogen-free water, before use. Alteratively, a body area may be swabbed, sprayed or applied with the drugs.

The drugs may also be formulated as a rectal drug such as suppositories or retention enemas, e.g., containing conventional suppository bases such as cocoa butter or other glycerides.

The drugs may also be formulated as a depot preparation. For example, parenteral depot systems (PDS) are injected or implanted into the muscle or subcutaneous tissue and incorporated drug released in a controlled manner, allowing the adjustment of release rates over extended periods of time, ranging from several days up to one year. Such long acting formulations may be administered by implantation (e.g., subcutaneously or intramuscularly) or by intramuscular injection. The drugs may be formulated with suitable polymeric or hydrophobic materials (e.g., as an emulsion in an acceptable oil) or ion exchange resins, or as sparingly soluble derivatives, such as a sparingly soluble salt. Other suitable delivery systems include microspheres which offer the possibility of local noninvasive delivery of drugs over an extended period of time. This technology utilizes microspheres of precapillary size which can be injected via a coronary catheter into any selected part of the body without causing inflammation or ischemia. The administered drug is slowly released from these microspheres and taken up by surrounding tissue cells.

Systemic administration of the drugs can also be by transmucosal or transdermal means. For transmucosal or transdermal administration, penetrants appropriate to the barrier to be permeated are used in the formulation. Such penetrants are generally known in the art, and include, e.g., for transmucosal administration bile salts and fusidic acid derivatives. In addition, detergents may be used to facilitate permeation. Transmucosal administration may be through nasal sprays or using suppositories. For topical administration, the oligomers of the invention are formulated into ointments, salves, gels, or creams as generally known in the art. A wash solution may be used locally to treat an injury or inflamination to accelerate healing.

Practice of the invention can be understood from the following examples, which are presented herein for illustration only and should not be construed as limiting the invention in any way.

### EXAMPLES

### Example 1

### Clinical Trial

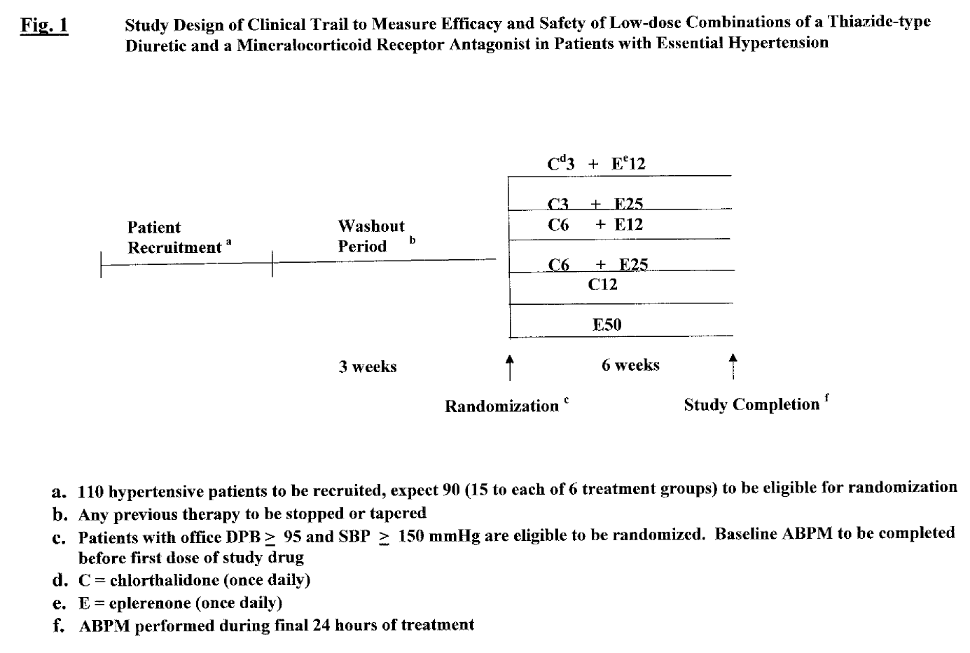

The efficacy and tolerability of a low dose (6-25 mg) eplerenone and (3-12 mg) chlorthalidone drug combination can be measured to demonstrate enhanced antihypertensive efficacy compared to currently available full doses of single agent therapy with either chlorthalidone (25 mg daily) or spironolactone (25 mg daily). See FIG. 1.

### INCORPORATION BY REFERENCE

The contents of all cited references (including literature references, patents, patent applications, and websites) that maybe cited throughout this application are hereby expressly incorporated by reference. The practice of the present invention will employ, unless otherwise indicated, conventional techniques of drug manufacture and administration, which are well known in the art.

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

1. A pharmaceutical composition comprising analdosterone antagonist and athiazide in which
(a) the aldosterone antagonist and thiazide act synergistically;
(b) the aldosterone antagonist and thiazide provide potassium neutrality; or
(c) the aldosterone antagonist and thilazide each provide ail equal contribution to an antihypertensive effect.

2. The pharmaceutical composition of claim 1 wherein said aldoster one antagonist and said thiazide are formulated in separate dosage forms which are co-packaged.

3. The pharmaceutical composition of claim 1 wherein said aldosterone antagonist and said thiazide are co-formulated in a single dosage form.

4. The pharmaceutical composition of claim 1 wherein said aldosterone antagonist and said thiazide are formulated in separate dosage forms which are co-packaged.

5. The pharmaceutical composition of claim 1 wherein said aldosteroneo antagonist is selected from the group consisting of spironotactone and eplerenone.

6. The pharmaceutical composition of claim 1 wherein said thiazide is selected from the group consisting of chlorthiatidone and hydrochlorothiazide.

7. A method of treatment with an aldosterone antagonist and a thiazide-type diuretic, said method providing a desired effect selected from the group consisting of
(a) synergism of thealdosterone antagonist and thiazide;
(b) fully additive effect by each of the aldosterone antagonist and thiazide;
(c) potassium neutrality provided by the aldosterone antagonist and thiazide; and
(d) equal contribution to antihypentensive effect by each of the aldosterone antagonist and thiazide drugs.

8. The method of claim 7 in which synergy or full additivity relate to amount or duration of anti-hypertensive effect.

9. The method of claim 7 in which potassium neutrality relates to serum or plasma potassium levels in a patient.

10. The method of claim 9 in which the patient is not receiving concomitant additional medication with effects on serum or plasma potassium levels.

11. The method of claim 9 in which the patient is receiving concomitant additional medication with effects on serum or plasma potassium levels.

12. The method of claim 7 in which the desired clinical endpoint comprises equal contribution to a hemodynamic effect.

13. The method of claim 12 in which said endpoint comprises anti-hypertensive effect.

14. The method of claim 7 in which the aldosterone antagonist is selected from the group consisting of spironolactone and eplerenone.

15. The method of claim 7 in which the thiazide-type diuretic is selected from the group consisting of chlorthalidone and hydrochlorothiazide.

16. The method of claim 7 in which the aldosterone antagonist and the thiazide-type diuretic are co-administered.

17. The method of claim 7 in which the aldosterone antagonist and the thiazide-type diuretic are given during the same day.

18. The method of claim 7 in which the aldosterone antagonist and the thiazide-type diuretic are given on alternate days.

19. The method of claim 7 in which the eplerenone daily dose is from about 5 to about 50 mg.

20. The method of claim 7 in which the hydrochlorothiazide daily dose is from about 6.25 to about 25 mg.

21. The method of claim 16 in which said thiazide-type diuretic is chlorthalidone and is used in a daily dose of from about 5 to about 25 mg.
